(19) United States
(12) Patent Application Publication
(10) Pub. No.: US 2021/0030870 A1
(43) Pub. Date: Feb. 4, 2021

(54) MODULATION OF P62 AND STING ACTIVITY

(71) Applicant: STipe Therapeutics ApS, Abyhoj (DK)

(72) Inventors: Thaneas Prabakaran, Risskov (DK); Søren Riis Paludan, Åbyhøj (DK); Martin Roelsgaard Jakobsen, Risskov (DK)

(73) Assignee: STipe Therapeutics ApS, Abyhoj (DK)

(21) Appl. No.: 16/967,703

(22) PCT Filed: Feb. 6, 2019
(86) PCT No.: PCT/EP2019/052886

§ 371 Date: Aug. 5, 2020

(30) Foreign Application Priority Data
Feb. 6, 2018 (EP) 18155309.0

(51) Int. Cl. A61K 39/395; C07K 14/47; C07K 16/24; A61K 45/06; A61K 38/19; A61P 31/04

(52) U.S. Cl. CPC A61K 39/3955; C07K 14/47; A61P 31/04; A61K 45/06; A61K 38/19; C07K 16/24

(57) ABSTRACT

The present invention relates to tools for modulating the P62-STING interaction as a method for modulating the innate immune response. The invention relates to compounds which are capable of either inhibiting or potentiating the interaction and thereby induce or prevent the innate immune response relating to STING. Specifically, compounds are provided for use in the treatment of disorders associated with STING activity, including cancer and immuno-deficient or auto-immune disorders.

### TECHNICAL FIELD

The present invention relates to tools for modulating the P62-STING interaction as a method for modulating the innate immune response. The invention relates to compounds which are capable of either inhibiting or potentiating the interaction and thereby induce or prevent the innate immune response relating to STING. Specifically, compounds are provided for use in the treatment of disorders associated with STING activity, including cancer and immuno-deficient or auto-immune disorders.

### BACKGROUND

Stimulus-dependent activation of immune responses is essential to achieve optimal tempo-spatial immune activities at sites of infection and to avoid tissue damage. Innate immune activation by cytosolic DNA from microbial pathogens is a potent trigger of type I Interferon (IFN) and pro-inflammatory cytokines. The pathway that leads to IFN activation has been extensively studied both in terms of the proteins binding cytosolic DNA and those needed for subsequent downstream signaling and immune activation.

Cytosolic DNA is highly immune-stimulatory and is detected by a panel of sensors to stimulate production of type I interferon (IFN)s, interleukin 1β, and activation of death pathways. Cyclic GMP-AMP (cGAMP) synthase (cGAS) is the key cytosolic DNA sensor that stimulates the IFN induction pathway. Upon DNA binding, cGAS synthesizes 2′3′ cGAMP, which binds the adaptor protein Stimulator of IFN genes (STING) and induces a conformational change in the STING dimer, thus enabling recruitment and activation of the Tank-binding kinase (TBK)1 and the transcription factor IFN regulatory factor (IRF). The induction of type I IFNs by DNA is essential for defense against virus infections and for anti-cancer immunity.

Negative regulation of immune pathways is, however, essential to achieve resolution of immune responses and to avoid excess inflammation. Pathological roles have been ascribed to type I IFNs in bacterial infections and in chronic viral infections. Most notably, excess activation of the cGAS-STING pathway, is associated with autoinflammatory diseases, including Aicardi-Goutieres Syndrome and systemic lupus erythematosus.

Due to the central role of STING in inducing IFN in innate immune responses, methods of modulating STING activity are therefore highly desirable in order to be able to control the immune responses during infections and/or auto-immune diseases.

### SUMMARY

The present invention relates to the discovery that protein P62 is a key negative regulator of STING and provides autophagosomal maturation in order to degrade STING. P62 is a ubiquitin-binding selective autophagy receptor. Upon exposure of STING to cytoplasmic dsDNA, it is redistributed to P62-positive punctate structures in the cytoplasm (“sequestosomes”) that colocalize with autophagy-related proteins. There, P62 serves as a platform for the K63 polyubiquitination of STING that destines it for the degradation in lysosomal/autophagosomal compartments.

Modulation of the P62-STING interaction may therefore serve as a method for regulation of the innate immune response. Inhibition of the interaction may serve as a method of boosting the innate immune response, whereas potentiating the interaction may serve as a tool for controlling auto-immune diseases.

The present invention further relates to identification of compounds which are capable of modulating the interaction between STING and P62.

In one aspect, the present invention provides compounds which are capable of inhibiting or reducing the interaction between STING and P62. Thus, it is one aspect of the present invention to induce or enhance the activity of STING. It is a further aspect that the compounds are capable of inhibiting or reducing STING degradation.

In another aspect of the present invention, compounds which are capable of potentiating the interaction between STING and P62 are provided. Thus it is one aspect of the present invention to reduce the activity of STING. It is a further aspect that the compounds are capable of inducing or enhancing STING degradation.

In a third aspect, the invention provides molecules for use in the treatment of disorders associated with STING activity. Said disorder may be associated with insufficient STING activity. Said disorder may also be associated with excessive STING activity.

Said disorder may be cancer, infection with a DNA pathogen, an inflammatory disorder or an auto-immune disease.

### DESCRIPTION OF DRAWINGS

FIG. 1: Displays the localization of STING (left panel) and P62 (mid panel) in WT MEFs cells which have been stimulated with dsDNA. Scale bar 5 μM. A pronounced relocalization of P62 to the same areas as STING is observed.

FIG. 2: Displays the levels of IFNβ (A) and CXCL10 (B) in WT and p62−/− MEFs cells which have been stimulated with dsDNA or 2′3′ cGAMP. MEFs cells lacking P62 induced significantly elevated levels of IFNβ and ISGs after stimulation with dsDNA or cGAMP.

FIG. 3: Displays the level of CXCL10 in control and P62 KO THP1 cells which have been stimulated with dsDNA or 2′3′ cGAMP. THP1 cells lacking P62 induced expression of the ISG CXCL10 to a much higher extent than control cells after stimulation with dsDNA or cGAMP.

FIG. 4: Displays the levels of CXCL10 (A) and IFNβ (B) in human foreskin fibroblasts treated with control or P62-specific gRNA and subsequently stimulated with dsDNA. Western blots for P62 are shown to the right (C). Human foreskin fibroblasts responded with higher DNA-driven IFN/ISG expression after depletion of P62 expression.

FIG. 5: Displays the level of CXCL10 in WT and P62-deficient MEFs (A) and THP1 (B) cells as a function of time following stimulation with dsDNA. The elevated response in P62-deficient cells was more pronounced at later time points after stimulation, thus suggesting a role for P62 in negative feedback rather than constitutive control.

FIG. 6: Displays the degradation of STING in WT and p62 KO THP1 cells which have been stimulated with dsDNA. The turnover of STING was abolished after DNA stimulation in P62-deficient THP1 cells.

FIG. 7: Displays the degradation of STING in WT and p62^{-/-} MEFs cells which have been stimulated with. The turnover of STING was delayed and higher basal levels of STING was observed in p62^{-/-} MEFs.

FIG. 8: Displays the effect of reconstitution of P62 expression in P62 KO THP1 cells and p62−/− MEFs stimulated with dsDNA. The STING level was analyzed in lysates of THP1 cells (A) and CXCL10 levels analyzed in the supernatants of THP1 cells (B) and MEFs cells (C). The data are presented as means of 2 replicates +/−st.dev. *, 0.01<p<0.05; **, 0.001<p<0.005; ***, p<0.001. Reconstitution of wt P62 expression in P62-deficient MEFs and THP1 cells enabled these cells to degrade STING after DNA stimulation and reduced DNA-induced CXCL10 expression.

FIG. 9: Displays the effect of P62 on ubiquitination of STING in HEK293T cells which have been transduced with TRIM56, STING-HA, and P62-FLAG. The samples were processed using anti-HA immunoprecipitation. The precipitate was immunoblotted with anti-HA, anti-FLAG, and anti-ubiquitin. Elevated ubiquitination of STING was observed and the STING ubiquitination was accompanied by elevated P62-STING co-immunoprecipitation.

FIG. 10: Displays the level of CXCL10 in WT and p62^{-/-}MEFs which have been infected with Listeria monocytogenes (MOI 25) or MCMV (MOI 10). The data are presented as means of 3-5 replicates +/− st.dev. *, 0.01<p<0.05; **, 0.001<p<0.005; ***, p<0.001. Infections with the DNA pathogens stimulated elevated responses in the P62-deficient cells.

FIG. 11: Displays the level of CXCL10, P62 and STING in control and P62 KO THP1 cells which were infected with HSV-1 (MOI 10) for 0, 3, 6, 12, 18 or 24 hours. Supernatants were analyzed for levels of CXCL10 and lysates were immunoblotted for P62, STING, phospho-TBK1, and 13-actin. The data are presented as means of 3-5 replicates +/− st.dev. *, 0.01<p<0.05; **, 0.001<p<0.005; ,***, p<0.001. Infection with HSV-1 induced a strongly elevated production of CXCL10 in P62-deficient cells compared to the control cells, and STING was not degraded in the infected knockout cells.

FIG. 12: Displays the level of type I IFN, P62 and STING in human monocyte-derived macrophages wherein P62 was knocked down by siRNA. The siRNA-treated cells were treated with dsDNA or infected with HSV-1. Supernatants were analyzed for levels of type I IFN at time point 12 hours post treatment, and lysates were immunoblotted for P62 and STING at timepoints 0 or 12 hours post treatment. The data are presented as means of 3-5 replicates +/− st.dev. *, 0.01<p<0.05; **, 0.001<p<0.005; ***, p<0.001. A strong knockdown of P62 in the primary human macrophages was achieved, and it was observed that cells with reduced P62 expression produced more type I IFN after stimulation with DNA or infection with HSV-1. Higher constitutive levels of STING and impaired DNA-induced degradation of STING was observed.

FIG. 13: Illustration of P62 domains and the specific peptides derivatives from the P62 amino acid sequence. The figure includes peptides fragments of the N-terminus of P62 (SEQ ID NO:5), the PB1 domain (SEQ ID NO:6); and ZZ domain (SEQ ID NO:7).

FIG. 14: Displays the results of a P62 targeting peptide with immunostimulatory effects. Pre-activation of human primary peripheral blood mononuclear cells (PBMCs) with specific P62 targeting peptide. The results shows that peptide SEQ ID NO:9 (PEP305 or 305) is able to enhance the cytokine response of TNF-alpha (panel A) or suppress type I Interferon (panel B) after co-stimulation with cGAMP (white) or Herring testis DNA (HT-DNA, black). Furthermore, in human PMA-differentiated THP1 cells, peptide SEQ ID NO: 9 also demonstrate the capacity to increase CXCL10 expression after HT-DNA treatment (panel C).

FIG. 15: Displays P62 targeting peptides with immunosuppressive effects. Pre-activation with peptide SEQ ID NO:8 (PEP300) and peptide SEQ ID NO:10 (PEP307) followed by co-stimulation with HT-DNA results in significant suppression of type I IFN in both PBMCs (panel A and B) and THP1 cells (panel C).

FIG. 16: Panel A: THP1 cells stimulated with DNA or polylC with/without PEP302 (Sp62-302, SEQ ID NO: 12) followed by type I IFN measurements by IFN bioassay. PEP302 may specifically target the DNA-cGAS STING pathway and not the polylC pathway. Panel B: CXCL10 ELISA data further support that PEP302 can inhibit DNA-STING-induced CXCL10 production. Panel C: Western blotting data show that PEP302 may inhibit phosphorylation of STING and consequently inhibit/reduce phosphorylation of downstream proteins such as TBK1 and p62 after DNA and cGAMP stimulation (inactivation of STING). Panel D: PEP302 seems to constitutively induce autophagy, induce p62 dimerization and potentially ubiquitination of STING (inactivation of STING).

FIG. 17: Biacore data that show PEP301 (Sp62-301, SEQ ID NO: 11) binds to STING with high affinity (Kd=746 nM).

### DETAILED DESCRIPTION

Definitions

The term “comprising” should be understood in an inclusive manner. Hence, by way of example, a composition comprising compound X, may comprise compound X and optionally additional compounds.

The term “polypeptide” as used herein refers to a chain of amino acid monomers linked by peptide (amide) bonds. Said chain may comprise any number of amino acid monomers, but typically comprise at least 5 amino acids. The polypeptide may comprise any amino acid, however preferably consists of naturally occurring amino acids.

The term “small organic molecules or compounds” refers herein to non-oligomeric, carbon containing compounds producible by chemical synthesis and generally having a size of less than 600 mass units.

Compound capable of modulating the interaction of P62 and STING STING is a protein which is part of the innate immune response and involved in inducing an interferon and pro-inflammatory cytokine response during infection. In the present invention, the protein P62 has been identified as being involved in negative regulation of STING, and modulation of the P62-STING interaction may therefore serve as a method for regulation of the innate immune response. Inhibition of the interaction may serve as a method of boosting the innate immune response, whereas potentiating the interaction may serve as a tool for controlling auto-immune diseases.

The invention thus provides tools for modulating the P62-STING interaction as a method for modulating the innate immune response.

In one embodiment, a compound which is capable of modulating the interaction between P62 and STING is provided. Said compound may be capable of modulating the interaction by binding to either P62 or STING in a manner to inhibit the interaction between the two. Alternatively, the compounds may be able to bind the STING-P62 complex and thereby stabilize the complex. The compound may also be able to bind either P62 or STING in a manner to modify the ability of either protein to form a homodimer and thus modulate the interaction of P62 and STING. The compounds may be further able to bind a third molecule and thereby stabilize or inhibit the interaction between STING and P62. Such compounds may herein also be referred to as “compounds of the invention”.

In one embodiment, the compound is capable of inhibiting the interaction between P62 and STING.

In one embodiment, the compound is capable of inducing or enhancing STING activation.

In one embodiment, the compound is capable of inhibiting or reducing the degradation of STING.

In another embodiment, the compound is capable of potentiating the interaction between P62 and STING.

In one embodiment, the compound is capable of inhibiting STING activation.

In one embodiment, the compound is capable of inducing or enhancing the degradation of STING.

The compound of the invention may be any kind of compound. In one embodiment the compound is a small molecule. The small molecule may in particular be a small organic molecule. Typically, small molecules, such as small organic molecules are molecules of 600 mass units or less.

In another embodiment the compound is a polypeptide. Polypeptides capable of modulating the STING activity may be identified in any useful manner. Non-limiting examples of methods for identifying polypeptides capable of modulating STING activity include phage display, phage-display peptide biopanEning; pull-down binding competition assays; Fluorescent Resonance Energy Transfer assay (FRET);

Biacore analysis; or Database/Bioinformatics based methods.

In one embodiment, the compound is an antibody, an antigen-binding fragment of an antibody or a synthetic antibody.

The antibody may be any antibody. For example, the antibody may be a naturally occurring antibody or a functional homologue thereof. A naturally occurring antibody is a heterotetrameric glycoproteins capable of recognizing and binding an antigen comprising two identical heavy (H) chains and two identical light (L) chains interconnected by disulfide bonds. Each heavy chain comprises or preferably consists of a heavy chain variable region (abbreviated herein as V_{H}) and a heavy chain constant region (abbreviated herein as C_{H}). Each light chain comprises or preferably consists a light chain variable region (abbreviated herein as V_{L}) and a light chain constant region (abbreviated herein as C_{L}). The V_{H} and V_{L} regions can be further subdivided into regions of hypervariability, termed complementarity determining regions (CDRs), interspersed with regions that are more conserved, termed framework regions (FRs).

The naturally occurring antibody may also be a heavy-chain antibody (HCAbs) as produced by camelids (camels, dromedaries and llamas). HCAbs are homodimers of heavy chains only, devoid of light chains and the first constant domain (Hamers-Casterman et al., 1993).

The naturally occurring antibody according to the invention may for example be selected from the group consisting of IgG, IgM, IgA, IgD and IgE. The subunit structures and three-dimensional configurations of these different classes of immunoglobulins are well known.

Naturally occurring antibodies according to the invention may be antibodies of a particular species, for example the antibody may be a murine, a rat, a rabbit, a goat, a sheep, a chicken, a donkey, a camelid or a human antibody. The antibody according to the invention may however also be a hybrid between antibodies from several species, for example the antibody may be a chimeric antibody, such as a humanised antibody.

The antibody according to the invention may be a monoclonal antibody, such as a naturally occurring monoclonal antibody or it may be polyclonal antibodies, such as naturally occurring polyclonal antibodies.

The antigen binding fragment of an antibody may be any protein or polypeptide containing an antigen binding site. Preferably, the antigen binding site comprises at least one CDR, or more preferably a variable region

Thus the antigen binding site may comprise a V_{H} and/or V_{L}. It is preferred that the antigen binding site comprises one or more CDRs, preferably at least 1, more preferably at least 2, yet more preferably at least 3, even more preferably at least 4, yet more preferably at least 5, even more preferably 6 CDRs. It is preferable that the antigen binding site comprises at least one CDR3, more preferably at least the CDR3 of the heavy chain.

The antigen binding fragment of antibody may also be a heterospecific antibody, a single chain antibody or a recombinant antibody. The fragments may also be Fab fragments or scFv.

The antibody may be a synthetic antibody. Synthetic antibodies may for example be recombinant antibodies, nucleic acid aptamers and non-immunoglobulin protein scaffolds.

Recombinant antibodies may be generated in vitro by expression from recombinant genes. The recombinant genes may be based on antibody genes from any species of antibody-producing animal, which optionally may be manipulated to generate new antibodies or antibody fragments, such as Fab fragments and scFv. Synthetic antibodies may also be non-immunoglobulin derived. Such molecules typically differ in structure to that of an antibody and can for example be generated from nucleic acids, as in the case of aptamers, or from protein scaffolds, for example peptide aptamers, into which hypervariable loops are inserted to form the antigen binding site.

The synthetic antibody may also be an affimer protein, which is a small robust affinity reagents with a molecular weight of 12-14 kDa. Affimers are engineered to bind to their target proteins with high affinity and specificity. The Affimer protein scaffold is derived from the cysteine protease inhibitor family of cystatins, which contains two variable peptide loops and a variable N-terminal sequence, which can be engineered to provide a high affinity binding surface.

In one embodiment, the compound further comprises at least one conjugated moiety. Said at least one conjugated moiety may be a peptide, a sugar, a lipid, a cell-penetrating peptide (CPP) or any other chemical group that can be covalently linked to a polypeptide. The conjugated moiety may also improve physical properties of the polypeptide, such as its solubility, stability or half-life.

In one embodiment, the conjugated moiety may be a compound that masks the polypeptide from the host immune system, such as a polyethylene glycol (PEG) polymer chain or a modified PEG, for example NPEG.

The compound of the invention may be any compound which is capable of binding to P62. It is preferred that the compound is capable of selectively binding P62 and thus said compound preferably binds P62 with at least 10 times higher affinity than to a non-specific polypeptide (e.g. BSA). It may further be preferred that said compound binds P62 with higher affinity, e.g, with at least 2× higher affinity than it binds to any other polypeptide.

In some embodiments the compound may be capable of binding P62 or a fragment thereof with an affinity corresponding to a K_{D} of about 10⁻⁷ M or less, such as about 10⁻⁸ M or less, such as about 10⁻⁹ M or less, for example about 10⁻¹⁰ M or less, or even about 10⁻¹¹ M or even less.

The compound of the invention may be any compound which is capable of binding to STING. It is preferred that the compound is capable of selectively binding STING, and thus said compound preferably binds STING with at least 10 times higher affinity than to a non-specific polypeptide (e.g. BSA). It may further be preferred that said compound binds STING with higher affinity, e.g, with at least 2× higher affinity than it binds to any other polypeptide.

In some embodiments the compound may be capable of binding STING or a fragment thereof with an affinity corresponding to a KD of about 10⁻⁷ M or less, such as about 10 M or less, such as about 10⁻⁹ M or less, for example about 10⁻¹⁰ M or less, or even about 10⁻¹¹ M or even less.

Inhibition of P62-STING Interaction

The present invention provides peptides which are capable of inhibiting the interaction between P62 and STING and thus inducing or enhancing the activity of STING and/or inhibiting or reducing the degradation of STING.

In one embodiment, the compound is capable of mimicking the N-terminal domain of P62. In one embodiment the compound is a polypeptide which comprises or consists of the N-terminal domain of P62 or a fragment thereof,

In one embodiment, the compound is a polypeptide comprising or consisting of
a) the N-terminal domain of P62 (human P62) provided herein as SEQ ID NO: 2;
b) a fragment of said human N-terminal domain of P62 consisting of a consecutive sequence of at least 5 amino acids of SEQ ID NO: 2; or
c) a functional homologue of human N-terminal domain of P62 sharing at least 70% sequence identity with SEQ ID NO: 2.

In one embodiment, the compound is a polypeptide comprising or consisting of
a) a peptide according to SEQ ID NO: 5;
b) a fragment of SEQ ID NO: 5 consisting of a consecutive sequence of at least 5 amino acids of SEQ ID NO: 5; or
c) a functional homologue of SEQ ID NO: 5 sharing at least 70% sequence identity with SEQ ID NO: 5.

Said polypeptides or fragments or homologues thereof may further comprise at least one conjugated moiety. Said moiety may for example be a cell-penetrating peptide, such as for example polyarginine or TAT.

Thus, in one embodiment, the compound is a polypeptide comprising or consisting of
a) a peptide according to SEQ ID NO: 8;
b) a fragment of SEQ ID NO: 8 consisting of a consecutive sequence of at least 5 amino acids of SEQ ID NO: 8; or
c) a functional homologue of SEQ ID NO: 8 sharing at least 70% sequence identity with SEQ ID NO: 8.

In another embodiment, the compound is capable of mimicking the ZZ finger domain of P62. In one embodiment the compound is a polypeptide which comprises or consists of the ZZ finger domain of P62 or a fragment thereof,

In one embodiment, the compound is a polypeptide comprising or consisting of
d) the ZZ finger domain of P62 (human ZZ finger domain) provided herein as SEQ ID NO: 4;
e) a fragment of said human ZZ finger domain consisting of a consecutive sequence of at least 5 amino acids of SEQ ID NO: 4; or
f) a functional homologue of the human ZZ finger domain sharing at least 70% sequence identity with SEQ ID NO: 4.

In one embodiment, the compound is a polypeptide comprising or consisting of
d) a peptide according to SEQ ID NO: 7;
e) a fragment of SEQ ID NO: 7 consisting of a consecutive sequence of at least 5 amino acids of SEQ ID NO: 7; or
f) a functional homologue of SEQ ID NO: 7 sharing at least 70% sequence identity with SEQ ID NO: 7.

Said polypeptides or fragments or homologues thereof may further comprise at least one conjugated moiety. Said moiety may for example be a cell-penetrating peptide, such as for example polyarginine or TAT.

Thus, in one embodiment, the compound is a polypeptide comprising or consisting of
a) a peptide according to SEQ ID NO: 10;
b) a fragment of SEQ ID NO: 10 consisting of a consecutive sequence of at least 5 amino acids of SEQ ID NO: 10; or
c) a functional homologue of SEQ ID NO: 10 sharing at least 70% sequence identity with SEQ ID NO: 10.

Potentiation of P62-STING Interaction

The present invention provides compounds which are capable of potentiating the interaction between P62 and STING and thus inhibiting the activity of STING and/or inducing or enhancing the degradation of STING.

In one embodiment the compound is a polypeptide which comprises or consists of the PB1 domain of P62 or a fragment thereof.

In one embodiment, the compound is a polypeptide comprising or consisting of
a) the PB1 domain of P62 (human PB1 domain) provided herein as SEQ ID NO: 3;
b) a fragment of said human PB1 domain consisting of a consecutive sequence of at least 5 amino acids of SEQ ID NO: 3; or
c) a functional homologue of the human PB1 domain sharing at least 70% sequence identity with SEQ ID NO: 3.

In one embodiment, the compound is a polypeptide comprising or consisting of
a) a peptide according to SEQ ID NO: 6;
b) a fragment of SEQ ID NO: 6 consisting of a consecutive sequence of at least 5 amino acids of SEQ ID NO: 6; or
c) a functional homologue of SEQ ID NO: 6 sharing at least 70% sequence identity with SEQ ID NO: 6.

In another embodiment, the compound is a polypeptide comprising or consisting of
a) a peptide according to SEQ ID NO: 11;
b) a fragment of SEQ ID NO: 11 consisting of a consecutive sequence of at least 5 amino acids of SEQ ID NO: 11; or
c) a functional homologue of SEQ ID NO: 11 sharing at least 70% sequence identity with SEQ ID NO: 11.

In one embodiment, the compound is a polypeptide comprising or consisting of
a) a peptide according to SEQ ID NO: 12;
b) a fragment of SEQ ID NO: 12 consisting of a consecutive sequence of at least 5 amino acids of SEQ ID NO: 12; or c) a functional homologue of SEQ ID NO: 12 sharing at least 70% sequence identity with SEQ ID NO: 12.

Said polypeptides or fragments or homologues thereof may further comprise at least one conjugated moiety. Said moiety may for example be a cell-penetrating peptide, such as for example polyarginine or TAT.

Thus, in one embodiment, the compound is a polypeptide comprising or consisting of
a) a peptide according to SEQ ID NO: 9;
b) a fragment of SEQ ID NO: 9 consisting of a consecutive sequence of at least 5 amino acids of SEQ ID NO: 9; or
c) a functional homologue of SEQ ID NO: 9 sharing at least 70% sequence identity with SEQ ID NO: 9.

P62

P62 is a ubiquitin-binding selective autophagy receptor which is involved in degradation of STING, for example following activation of STING by extracellular DNA. In humans, P62 is encoded by the sqstml gene, and the amino acid sequence of human P62 is provided herein as SEQ ID NO: 1.

P62 contains several domains including a PB1 domain, a ZZ finger domain, a TBS domain, a LIR domain, a KIR domain and a UBA domain. An overview of the domain structure of P62 is provided herein in FIG. 13.

In one embodiment, the peptides provided herein for modulation of the interaction between P62 and STING comprises or consists of the N-terminal domain of P62, provided herein as SEQ ID NO: 2, or any fragment or analogue thereof.

In one embodiment, the peptides provided herein for modulation of the interaction between P62 and STING comprises or consists of the PB1 domain of P62, provided herein as SEQ ID NO: 3, or any fragment or analogue thereof.

In one specific embodiment, the peptides provided herein for modulation of the interaction between P62 and STING are selected from the group consisting of the SEQ ID NO: 6, 11, and/or 12.

In one embodiment, the peptides provided herein for modulation of the interaction between P62 and STING comprises or consists of the ZZ-finger domain of P62, provided herein as SEQ ID NO: 4, or any fragment or analogue thereof.

Polypeptides of present invention are preferably not too large. Accordingly it may be preferred that such polypeptide consists of at the most 150 amino acids, such as of the most 100 amino, for example at the most 80 amino acids.

Polypeptides of present invention may however also be a functional homologue of the N-terminal, PB1 or ZZ finger domains of P62 sharing at least 70%, such as at least 75%, for example at least 80%, such as at least 85%, for example at least 90%, such as at least 95%, for example at least 98% sequence identity with the human sequences provided herein as SEQ ID NOs: 2-4.

The invention also relates to fragments of the N-terminal, PB1 or ZZ finger domains of P62 as well as to compounds binding such fragments. Fragments of the N-terminal, PB1 or ZZ finger domains of P62 may be any fragment of any of the N-terminal, PB1 or ZZ finger domains of P62 described above. Typically, the fragments comprise at least 5 consecutive amino acids of the N-terminal, PB1 or ZZ finger domains of P62.

In one embodiment, the fragment comprise at least 5, such as at least 10, for example at least 15, such as at least 20, for example in the range of 5 to 70, such as in the range of 5 to 60, for example in the range of 5 to 50, such as in the range of 10 to 70, for example in the range of 10 to 60, such as in the range of 10 to 50 consecutive amino acids of the N-terminal, PB1 or ZZ finger domains of P62 provided herein as SEQ ID NOs: 2-4.

It may be preferred that aforementioned fragments of the N-terminal, PB1 or ZZ finger domains of P62 also retain one or more of the activities of P62 or STING described herein below in the section “P62 and STING activity”.

P62 and STING Activity

The invention generally relates to compounds capable of modulating the interaction between P62 and STING, thereby also being capable of regulating STING activity. The compounds are in one preferred embodiment capable of mimicking the ZZ finger domain or the PB1 domain of P62.

These compounds, such as peptides preferably retain one or more P62 activities.

The compounds may be capable of binding the ZZ finger or PB1 domain of P62, thereby inhibiting one or more P62 activities.

The inventors have shown that P62 is capable of interacting with the endoplasmic reticulum-bound protein stimulator of interferon genes (STING). Thus, in one preferred embodiment, the compounds provided herein are capable of interacting with STING, and serve as a negative regulator of STING activity. Thus, the compounds provided herein mimicking the ZZ finger or PB1 domain of P62 may be able to rescue STING from the negative regulation induced by binding of the native P62. In this way these compound are immune-stimulatory compounds.

Thus, in one preferred embodiment, the compounds mimicking the ZZ finger or PB1 domain of P62 are capable of inhibiting or at least reducing interaction between P62 and STING. Reduction of interaction is preferably at least a 2-fold reduction of the interaction. Interaction with STING may for example be determined by immunoprecipitation of P62 or fragments thereof using P62 antibodies, and subsequent detection of STING precipitating with P62 or fragments thereof, e.g. by Western blotting with antibodies to STING. The interaction may also be performed in the reverse manner, by immunoprecipitation of STING using antibodies to STING, and subsequent detection of P62 precipitating with STING, e.g. by Western blotting.

STING activation may be determined in a number of different ways including the following:

STING activation may be determined by determining STING phosphorylation. Thus, in one preferred embodiment, the compounds mimicking the ZZ finger or PB1 domain of P62 are capable of inducing phosphorylation of STING or the presence of phosphorylated STING, e.g inducing an at least 2 fold increase in phosphorylation of STING.

Conversely, the compounds provided herein, which are capable of potentiating the interaction between P62 and STING, are also preferably capable of inhibiting or at least reducing phosphorylation of STING, such as reducing phosphorylation of STING at least 2-fold. Said phosphorylation of STING may in particular be phosphorylation of Ser366 of STING.

STING activation may also be determined as activation of expression of type I IFN or inflammatory cytokines in cells capable of expressing type I IFN or cytokines. Examples of such cells include macrophages, dendritic cells, keratinocytes, fibroblasts, monocytes, epithelia cells, B cells, or NK cells. Thus, STING activation may be determined by determining expression of type I IFN or cytokines in such cells.

STING activation may also be determined as activation of IFNβ promoter activity. Activity of the IFNβ promoter may for example be determined in recombinant cells comprising a nucleic acid construct encoding a reporter protein under the control of the IFNβ promoter. IFNβ promoter can also be determined in cell free expression systems allowing expression of a reporter protein under the control of the IFNβ promoter.

Disorder Associated with STING Activity

In one embodiment the invention relates to compounds capable modulating the interaction of P62 and STING for use in the treatment of a disorder associated with STING activity. Said compounds may for example be any of the compounds described herein above in the section “Compound capable of modulating the interaction of P62 and STING”. In particular the compound may be any of the compounds as described herein.

The disorder associated with STING activity may for example be a disorder characterized with by excessive STING activity or by undesired STING activity. Said STING activity may for example be any of the activities described herein above in the section “P62 and STING activity”.

Numerous disorders have been associated with STING activity for example as described in any of the following references:
STING-mediated DNA sensing promotes antitumor and autoimmune responses to dying cells; Klarquist et al.; The Journal of Immunology, Vol. 193, Issue 12, 15 Dec. 2014 (6124-34).
STING Promotes the Growth of Tumors Characterized by Low Antigenicity via IDO Activation; Lemos H et al.; Cancer research, April 2016, Volume 76, Issue 8 (2076-81).
Intrinsic Self-DNA Triggers Inflammatory Disease Dependent on STING; Ahn et al.; The Journal of Immunology, Vol. 193, Issue 9, 1 Nov. 2014 (4634-4642).
STING Activation by Translocation from the ER Is Associated with Infection and Autoinflammatory Disease; Dobbs et al; Cell Host & Microbe, Volume 18, Issue 2, Aug. 12, 2015 (157-68).
Activation of cyclic GMP-AMP synthase by self-DNA causes autoimmune diseases; Gao et al; PNAS, Volume 112, no. 42, Oct. 20, 2015 (5699-705).
Therapeutic potential of targeting TBK1 in autoimmune diseases and interferonopathies; Hasan & Yan; Pharmacological Research, Volume 111, September 2016 (336-342).

In one embodiment of the invention the disorder associated with STING activity is cancer. In particular, said cancer may be a cancer induced by chronic inflammatory signaling. For example said cancer may be a cutaneous skin tumor, for example basal cell (BCC) or squamous cell carcinoma (SCC).

In one embodiment the disorder associated with STING activity is an inflammatory disorder. Said inflammatory disorder may for example be selected from the group consisting of psoriasis, Crohn's disease and Inflammatory bowel disease (IBD).

In one embodiment the disorder associated with STING activity is an auto-immune disease. Said autoimmune disease may for example be selected from the group consisting of Paget's disease, systemic lupus erythematosus (SLE), STING-associated vasculopathy with onset in infancy (SAVI), Aicardi-Goutieres syndrome, Sjogren's syndrome, Type 1 diabetes and multiple sclerosis.

The disorder may also be both an inflammatory disorder and an auto-immune disease. Thus, many auto-immune diseases are also inflammatory disorders.

Disorder Associated with Insufficient STING Activity

In one embodiment the invention relates to compounds capable modulating the interaction of P62 and STING for use in the treatment of a disorder associated with insufficient STING activity.

As demonstrated by the present invention, the compounds of the invention may induce STING activity. Accordingly, the compounds may be useful for treating disorders associated with insufficient STING activity.

In one embodiment the disorder is cancer. Cancer (malignant neoplasm) is a class of diseases in which a group of cells display the traits of uncontrolled growth (growth and division beyond the normal limits), invasion (intrusion on and destruction of adjacent tissues), and sometimes metastasis (spread to other locations in the body via lymph or blood). Most cancers form a tumor but some, like leukemia, do not.

Thus, the disorder may be cancer, for example a cancer selected from the group consisting of: colon carcinoma, breast cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangeosarcoma, lymphangeoendothelia sarcoma, synovioma, mesothelioma, Ewing's sarcoma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioblastomas, neuronomas, craniopharingiomas, schwannomas, glioma, astrocytoma, medulloblastoma, craniopharyngioma, ependymoma, pinealoma, hemangioblastoma, acoustic neuroama, oligodendroglioma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, childhood-null acute lymphoid leukemia (ALL), thymic ALL, B-cell ALL, acute myeloid leukemia, myelomonocytoid leukemia, acute megakaryocytoid leukemia, Burkitt's lymphoma, acute myeloid leukemia, chronic myeloid leukemia, and T cell leukemia, small and large non-small cell lung carcinoma, acute granulocytic leukemia, germ cell tumors, endometrial cancer, gastric cancer, cancer of the head and neck, chronic lymphoid leukemia, hairy cell leukemia and thyroid cancer.

The disorder may also be an infection with DNA pathogens, where IFN is deleterious. Such disorders include for example malaria or listeria.

Method of Treatment

As described herein the invention in some embodiments relates to compounds capable of modulating the interaction between P62 and STING for use in methods of treatment.

Whilst it is possible for the compounds or polypeptides of the present invention to be administered as the raw chemical, it is preferred to present them in the form of a pharmaceutical formulation. Accordingly, the present invention further provides a pharmaceutical formulation, which comprises a compound of the present invention or a pharmaceutically acceptable salt thereof, and a pharmaceutically acceptable carrier therefore. The invention also provides pharmaceutical formulations comprising a compound of the invention and a pharmaceutically acceptable carrier therefore.

The pharmaceutical formulations may be prepared by conventional techniques, e.g. as described in Remington: The Science and Practice of Pharmacy 2005, Lippincott, Williams & Wilkins.

The pharmaceutically acceptable carriers can be either solid or liquid. Solid form preparations include powders, tablets, pills, capsules, cachets, suppositories, and dispersible granules. A solid carrier can be one or more excipients, which may also act as diluents, flavoring agents, solubilizers, lubricants, suspending agents, binders, preservatives, wetting agents, tablet disintegrating agents, or an encapsulating material.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The compounds or polypeptides of the present invention may be formulated for parenteral administration and may be presented in unit dose form in ampoules, pre filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents.

Alternatively, the active ingredient may be in powder form, obtained by aseptic isolation of sterile solid or by lyophilisation from solution for constitution before use with a suitable vehicle, e.g., sterile, pyrogen-free water.

Preferably, the formulation will comprise about 0.5% to 75% by weight of the active ingredient(s) with the remainder consisting of suitable pharmaceutical excipients as described herein.

Pharmaceutically acceptable salts of the compounds, where they can be prepared, are also intended to be covered by this invention. These salts will be ones that are acceptable in their application to a pharmaceutical use. Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds or polypeptides of the invention are in general administered in an “effective amount” or an amount necessary to achieve an “effective level” in the individual patient. When the “effective level” is used as the preferred endpoint for dosing, the actual dose and schedule can vary, depending on inter-individual differences in pharmacokinetics, drug distribution, and metabolism. The “effective level” can be defined, for example, as the blood or tissue level desired in the patient that corresponds to a concentration of the compounds or polypeptides according to the invention.

The compounds or polypeptides of the invention may be administered together with one or more other active compounds, typically with one or more other active compounds useful for treatment of the particular disorder to be treated. Thus, in embodiments of the invention, wherein the disorder is cancer, the compounds or polypeptides of the invention may be administered together with one or more anticancer agents.

Sequences
Full length human P62
SEQ ID NO: 1
MASLTVKAYLLGKEDAAREIRRFSFCCSPEPEAEAEAAAGPGPCER
LLSRVAALFPALRPGGFQAHYRDEDGDLVAFSSDEELTMAMSYVKD
DIFRIYIKEKKECRRDHRPPCAQEAPRNMVHPNVICDGCNGPVVGT
RYKCSVCPDYDLCSVCEGKGLHRGHTKLAFPSPFGHLSEGFSHSRW
LRKVKHGHFGWPGWEMGPPGNWSPRPPRAGEARPGPTAESASGPSE
DPSVNFLKNVGESVAAALSPLGIEVDIDVEHGGKRSRLTPVSPESS
STEEKSSSQPSSCCSDPSKPGGNVEGATQSLAEQMRKIALESEGRP
EEQMESDNCSGGDDDWTHLSSKEVDPSTGELQSLQMPESEGPSSLD
PSQEGPTGLKEAALYPHLPPEADPRLIESLSQMLSMGFSDEGGWLT
RLLQTKNYDIGAALDTIQYSKHPPPL,
N-terminal domain of human P62
SEQ ID NO: 2
MASLTVKAYLLGKEDAARE,
PB1 domain of human P62
SEQ ID NO: 3
IRRFSFCCSPEPEAEAEAAAGPGPCERLLSRVAALFPALRPGGFQA
HYRDEDGDLVAFSSDEELTMAMSYVKDDIFRIYIKEK,
ZZ-domain of human P62
SEQ ID NO: 4
VHPNVICDGCNGPWGTRYKCSVCPDYDLCSVCEGKGLHRGHTKLA,
N-terminal fragment peptide of human P62
SEQ ID NO: 5
SLTVKAYLLGKEDAAREIRR,
PB1 domain fragment peptide of human P62
SEQ ID NO: 6
EELTMAMSYVKDDIFRIYIK,
ZZ domain fragment peptide of human P62
SEQ ID NO: 7
DLCSVCEGKGLHRGHTKLAF,
N-terminal fragment peptide of human
P62 C-terminally conjugated to TAT (PEP300)
SEQ ID NO: 8
SLTVKAYLLGKEDAAREIRRGRKKRRQRRRPQ,
PB1 domain fragment peptide of human
P62 C-terminally conjugated to TAT(PEP305)
SEQ ID NO: 9
EELTMAMSYVKDDIFRIYIKGRKKRRQRRRPQ,
ZZ domain fragment peptide of human
P62 C-terminally conjugated to TAT(PEP307)
SEQ ID NO: 10
DLCSVCEGKGLHRGHTKLAFGRKKRRQRRRPQ,
PB1 domain fragment peptide of human P62
(PEP301)
SEQ ID NO: 11
FSFCCSPEPEAEAEAAAGPG GRKKRRQRRRPQ,
PB1 domain fragment peptide of human P62
(PEP302)
SEQ ID NO: 12
PGPCERLLSRVAALFPALRP GRKKRRQRRRPQ,
P62 gRNA#1
SEQ ID NO: 13
GATGGCCATGTCCTACGTGA,
P62 gRNA#2
SEQ ID NO: 14
GTCATCCTTCACGTAGGACA,
STING_TMEM173 stimulator of interferon
genes protein isoform 1 [Homo sapiens]
SEQ ID NO: 15
MPHSSLHPSIPCPRGHGAQKAALVLLSACLVTLWGLGEPPEHTLRY
LVLHLASLQLGLLLNGVCSLAEELRHIHSRYRGSYWRTVRACLGCP
LRRGALLLLSIYFYYSLPNAVGPPFTWMLALLGLSQALNILLGLKG
LAPAEISAVCEKGNFNVAHGLAWSYYIGYLRLILPELQARIRTYNQ
HYNNLLRGAVSQRLYILLPLDCGVPDNLSMADPNIRFLDKLPQQTG
DHAGIKDRVYSNSIYELLENGQRAGTCVLEYATPLQTLFAMSQYSQ
AGFSREDRLEQAKLFCRTLEDILADAPESQNNCRLIAYQEPADDSS
FSLSQEVLRHLRQEEKEEVTVGSLKTSAVPSTSTMSQEPELLISGM
EKPLPLRTDFS

### EXAMPLE

### Example 1: Materials and methods

Reagents, virus and bacteria. For stimulations and inhibitions we used 60-mer dsDNA (DNA technology) (Unterholzner et al., 2010) or 2′3′cGAMP (InvivoGen). HSV-1(strain F) and Listeria monocytogenesis (strain L028) were propagated as described previously (Christensen et al., 2016; Hansen et al., 2014). For production of MCMV (strain K181), Ifnar^{-/-} MEFs were infected with virus at MOI 0.01. The infection was allowed to proceed until the cytopathic effects exceeded 90%, at which point the culture flasks were frozen. Cell debris was cleared from the thawed media by centrifugation at 4.500×g for 60 min (4° C.). The supernatant was subjected to centrifugation at 26.700×g on SORVAL RC6 plus (Thermo Scientific) for 5 hours, and the concentration of virus in the resuspended pellet was determined by titration on Ifnar^{-/-} MEF monolayers.

Cells. The cell lines used were MEFs, THP1, and human foreskin fibroblasts (HFFs). MEFs and HFFs were maintained in DMEM and THP1 cells were grown in RPMI 1640. All media were supplemented with 10% fetal calf serum and Penicillin-Streptomycin. The following MEF lines were used (laboratory which generated the line): p62^{-/-} (Masaaki Komatsu). The following genome-edited THP1-derived cell lines were used: P62 KO. See section below for details on how the cells were generated. For differentiation of monocyte-derived macrophages (MDM)s, Peripheral Blood Mononuclear cells were isolated from healthy donors by Ficoll Paque gradient centrifugation (GE Healthcare). Monocytes were separated by adherence to plastic in RPMI 1640 supplemented with 10% AB-positive human serum. Differentiation of monocytes to macrophages was achieved by culturing in Dulbecco's Modified Eagle Medium (DMEM) supplemented with 10% heat inactivated AB-positive human serum; 200 IU/mL Penicillin; 100 μg/mL Streptomycin and 600 pg/mL glutamine for 10 days in the presence of 10 ng/mL M-CSF (R&D Systems).

Generation of gene-modified cells. For generation of P62 deficient THP1 cells we constructed lentiviral vectors expressing Cas9 and gRNA using the lentiCRISPR v2 (Addgene plasmid # 52961). The gRNAs used were p62 gRNA#1, 5′-gATGGCCATGTCCTACGTGA-3′; p62 gRNA#2, 5′-GTCATCCTTCACGTAGGACA-3′. All gRNAs match sequences in the first exon of the target genes. The cells were transduced with the individual lentiviral vectors and diluted into single cells. The clones were expanded in the presence of puromycin (1 μg/mL) for 2 weeks and the complete knockout of the target gene in the clones was identified by Western blot. The p62 gRNA#1 lentiviral vector was also used to transduce human foreskin fibroblasts, in which case the transduced cells were treated with puromycin (1 μg/mL) for 30 days, and used for experiments without clonal selection. For rescue of P62 expression (wt or S403A) in P62 KO THP1 cells were transduced with lentivirus encoding human wt and S403S P62 modified in exon 1 to prevent targeting from gRNA#1 (Applied Biological Materials Inc.). Single cell clones were selected and expression of P62 was confirmed by Western blotting. For rescue of P62 expression in p62^{-/-} MEFs cells were transduced with high-titer lentivirus encoding murine wt P62 (Applied Biological Materials Inc.). The cells were incubated for 3 days and used for experiments.

siRNA Knock-Down in Primary Human Macrophages. For siRNA knock-down in MDMs, cells were transfected with a pool of P62 specific siRNAs (L-010230-00-0020) or scrambled siRNA controls (45 nM) using Lipofectamine RNAiMax (Life technologies) on days 6 and 8 into the differentiation procedure. The cells were used for experiments after 10 days of differentiation.

ELISA. CXCL10 protein levels were measured by ELISA using the hCXCL10/IP-10 DuoSet (R&D Systems).

Type I IFN Bioassay. Bioactive human type I IFN was measured on cell supernatants by use of HEK-Blue^{™}IFN-α/β cells as reporter cells according to the manufacturer instructions (InvivoGen), and as described by the manufacturer.

Immunoblotting. Whole-cell extracts or immunoprecipitation samples were analyzed by immunoblotting. Samples were diluted in XT sample buffer and XT reducing agent and ran on a SDS-PAGE (Criterion™ TGX™). Trans-Blot Turbo™ Transfer System® was used for the transfer of proteins to PVDF membranes (all reagents Bio-Rad). The membrane was blocked in 5% Difco™ skim milk (BD) or 5% bovine serum albumin (BSA) (Sigma). For detection of STING dimerization samples were prepared for electrophoresis using non-reducing RIPA lysis buffer with 0.2% SDS and complete mini inhibitor (Sigma). The antibodies used for Immunoblotting were: rabbit anti-cGAS (Cell Signaling, D1D3G/#15102, 1:1000), rabbit anti-STING (Cell Signaling, D2P2F/#13647, 1:1000), sheep anti-STING (R&D Systems, AF6516, 1:500), rabbit anti-pSTING (S366) (Cell SignalingTechnology, #85735), rabbit anti-TBK1 (Cell Signaling, D1B4/#3504, 1:1000), rabbit anti-pTBK1 (Ser172) (Cell Signaling, D52C2/#5483, 1:1000), rabbit anti-IRF3 (Cell Signaling, D83B9/#4302, 1:1000), rabbit anti-pIRF3 (Ser396) (Cell Signaling, D6O1M/#29047, 1:1000), guinea pig anti-P62 (Progen, GP62-C, 1:2000), rat anti-pP62 (Ser403) (MBL, D343-3, 1:1000), mouse anti-FLAG M2 (Sigma Aldrich, F3165, 1:1000), rabbit anti-HA (Cell Signaling, C29F4/#3724, 1:1000) and mouse anti-β-actin (Abca^{¬¬m, AC-}15/ab49900, 1:10000).

Extopic expression of proteins in HEK293T cells. HEK293T cells were seeded into 6 cm culture dishes to ˜70% confluency. The cells were transfected with 4 μg pRK-7-Flag-hSTING, and 4 μg HA-Ubiquitin (Addgene) using 12 μg polyethyleneimine (PEI). After transfection for 24 hours, the cells were lysed in IP-lysis buffer (Thermo Fisher Scientific) with 10 mM NaF and 1× Complete EDTA-free protease inhibitor cocktail tablets (Roche). The lysates were incubated with Anti-HA Magnetic Beads (Thermo Scientific) for 2 hours at room temperature. After 3 times washing, the immunoprecipitates were eluted. In another set of experiments, HEK293T cells stably expressing STING cells were transfected with 2 μg pIRESpuro3-P62-N3-FLAG using 2 μg PEI. Lysates and eluates were analyzed by Immunoblotting.

Immunoprecipitation. Cleared cell lysates were incubated with antibodies against STING (1:50) or P62 (1:50) overnight at 4° C. The antibodies used were rabbit anti-STING (Cell Signaling, D2P2F/#13647) and guinea pig anti-P62 (Progen, GP62-C). Protein G Dynabeads® (Novex by Life Technologies) were added for the last 2 h (0.9 mg per IP reaction in 240 μl). Immunoprecipitated complexes were eluted from the beads with a glycine buffer (200 mM glycine, pH 2,5), and protein expression was evaluated by Western blotting.

Purification of Endogenous Ubiquitin Conjugates. Tandem Ubiquitin Binding Entities (TUBEs) were used to purify endogenous ubiquitin conjugates from THP-1 cell lysates. Purification of Glutathione-S-transferase (GST) fused to 1×UBA^{ubq} Ub conjugates were pulled down in THP-1 cells using affinity reagents as in (Fiil et al., 2013). For isolation of K63-Ub chains and K48-Ub chains (LifeSensors) were used according to the manufacturer's recommendations. PMA differentiated 10×10⁶ THP-1 cells were lysed in ice-cold 600 μl TUBE lysis buffer contains (20 mM Na₂HPO₄, 20 mM NaH₂PO₄, 1% NP-40, 2 mM EDTA) supplemented with 1 mM DTT, 5 mM NEM (Sigma Aldrich), complete protease inhibitor cocktail (Roche), PhosSTOP (Roche). Pre-bound Glutathione Sepharose 4B beads (GE Healthcare) was added to 50 μg/mL of GST-TUBE for 1 hr. Lysates were incubated on ice and cleared by sonication and centrifugation, mixed the supernatant with beads followed the incubation at 4° C. for a minimum of 6 hours with continual rotation. The Sepharose beads were washed 3 times in 1 mL ice-cold PBS 0.1%. (v/v) Tween-20.1× LSB-buffer used to elute the bound Ub sample material.

Confocal microscopy. MEFs or PMA-differentiated THP1 cells were seeded on coverslips and stimulated as indicated. The cells were fixed with 4% formaldehyde (Sigma), permeabilized with 0.1% Triton X-100 (Sigma), and blocked with 1% BSA (Sigma). Cells were stained with primary antibodies for 2 h and with secondary antibody (all 1:500, Alexa Fluor)(Invitrogen) for 1 h. Images were acquired on Zeiss LSM 710 confocal microscope using a 63×1.4 oil-immersion objective and processed with the Zen2010 (Zeiss) and ImageJ software. The antibodies used were: sheep anti-STING (R&D Systems, AF6516, 1:50) and guinea pig anti-P62 (Progen, GP62-C, 1:200).

Statistics. For analysis of statistically significant difference between two groups of data we used 2-tailed Students t-test when the data exhibited normal distribution, and Wilcoxon rank sum test when the data set did not pass the normal distribution test.

### Example 2

### The Selective Autophagy Receptor P62/SQSTMI Exerts Negative Control of the cGAS-STING Pathway

The experiments were performed according to the general methods as described in Example 1 above.

The Localization of P62 and STING in MEFs Cells.

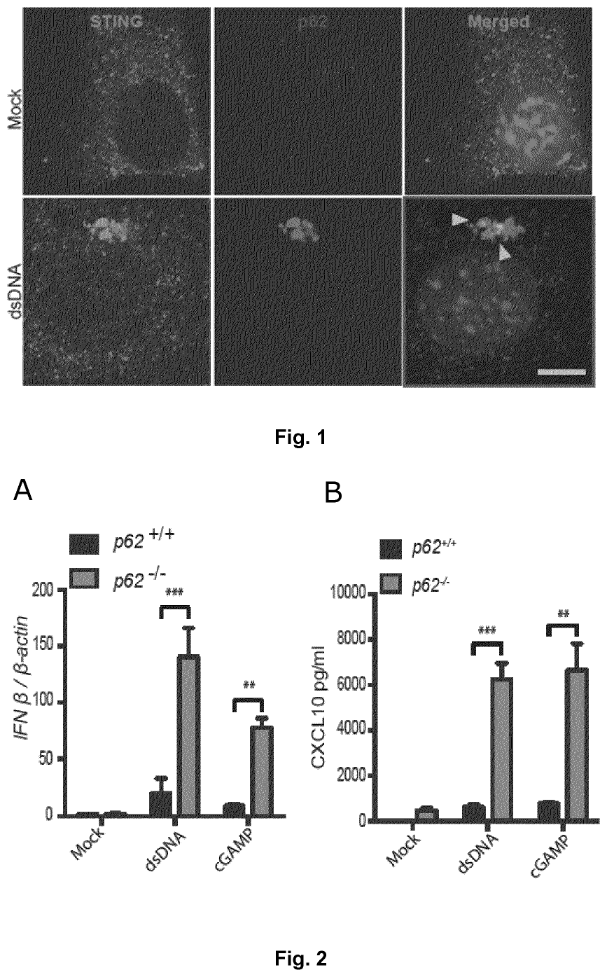

WT MEFs were stimulated with dsDNA (4 μg/ml). The cells were fixed 8 h later and stained with antibodies against total P62. A pronounced relocalization of P62 to the same areas as STING was observed upon stimulation (FIG. 1).

The Effect of P62 on IFNβ and CXCL10 Levels in MEFs Cells.

WT and p62^{-/-} MEFs were stimulated with dsDNA (4 μg/ml) or 2′3′ cGAMP (4 μg/ml). Total RNA and supernatants were harvested 6 and 18 h later, respectively, and levels of IFNβ and CXCL10 were measured. Significantly elevated levels of IFNβ and ISGs were observed in MEFs lacking P62 after stimulation with dsDNA or cGAMP (FIG. 2).

The Effect of P62 on CXCL10 Levels in THP1 Cells.

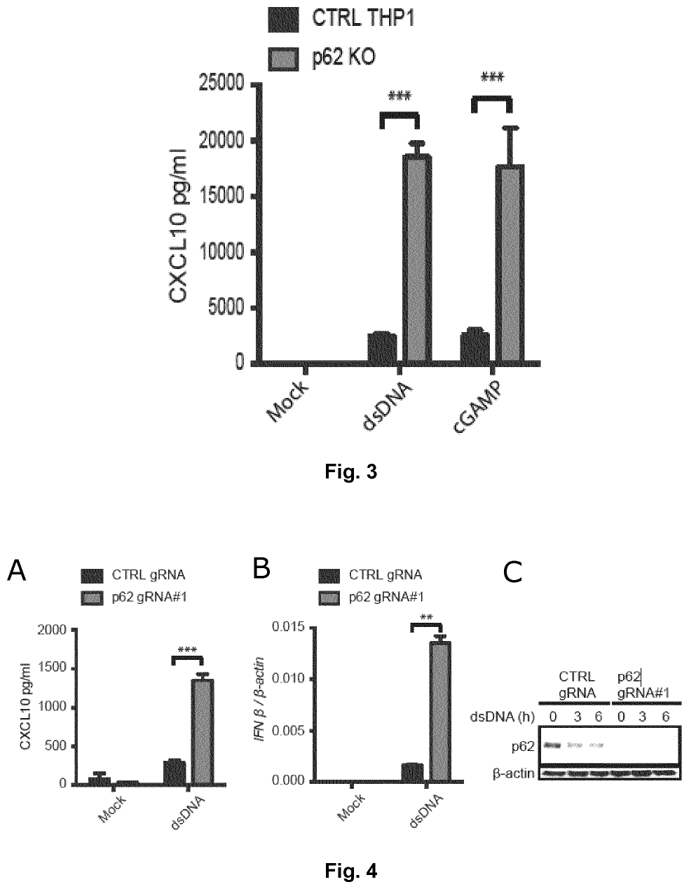

Control and P62 KO THP1 cells were stimulated with dsDNA (4 μg/ml) or 2′3′ cGAMP (4 μg/ml). Supernatants were harvested 18 h later, and levels of CXCL10 were measured. THP1 cells lacking P62 induced expression of the ISG CXCL10 to a much higher extent than control cells after stimulation with dsDNA or cGAMP (FIG. 3).

The effect of P62 on IFNβ and CXCL10 levels in Human foreskin fibroblasts. Human foreskin fibroblasts treated with control or P62-specific gRNA were stimulated with dsDNA (4 μg/ml). Total RNA and supernatants were harvested 6 and 18 h later, respectively, and levels of IFNβ and CXCL10 were measured. Western blots for P62 were obtained. Human foreskin fibroblasts responded with higher DNA-driven IFN/ISG expression after depletion of P62 expression (FIG. 4).

The Effect of P62 on CXCL10 Levels Over Time in MEFs and THP1 Cells.

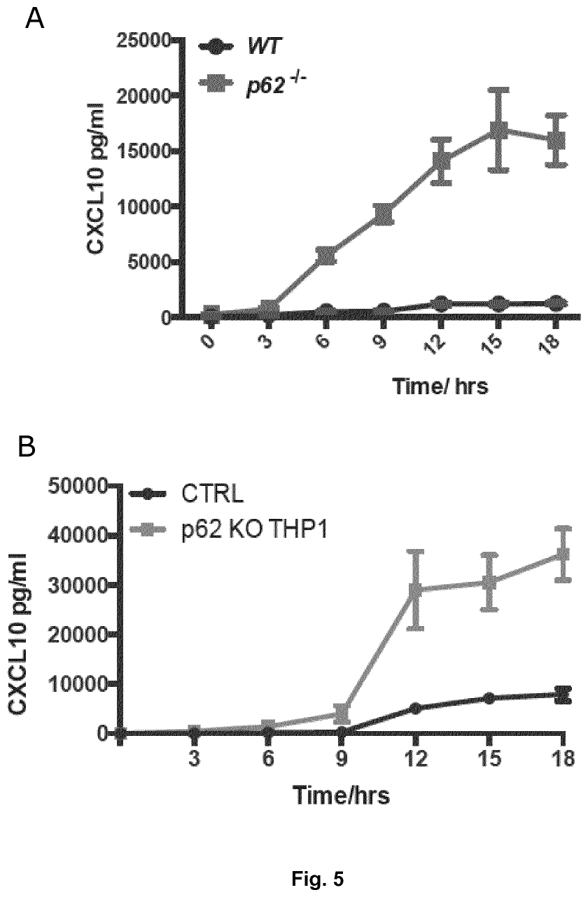

WT and P62-deficient MEFs and THP1 cells were stimulated with dsDNA (4 μg/ml). Supernatants were harvested at time points 0, 3, 6, 9, 12, 15 and 18 hours. Levels of CXCL10 were determined by ELISA. The data demonstrate that the elevated response in P62-deficient cells was more pronounced at later time points after stimulation, thus suggesting a role for P62 in negative feed-back rather than constitutive control (FIG. 5).

Overall, these experiments demonstrate that P62 interacts with STING and exerts negative control of the cGAS-STING pathway.

### Example 3

### STING is Degraded in a P62-Dependent Manner Correlating with its Ubiquitination

The experiments were performed according to the general methods as described in Example 1 above.

Effect of P62 on STING Degradation in THP1 Cells.

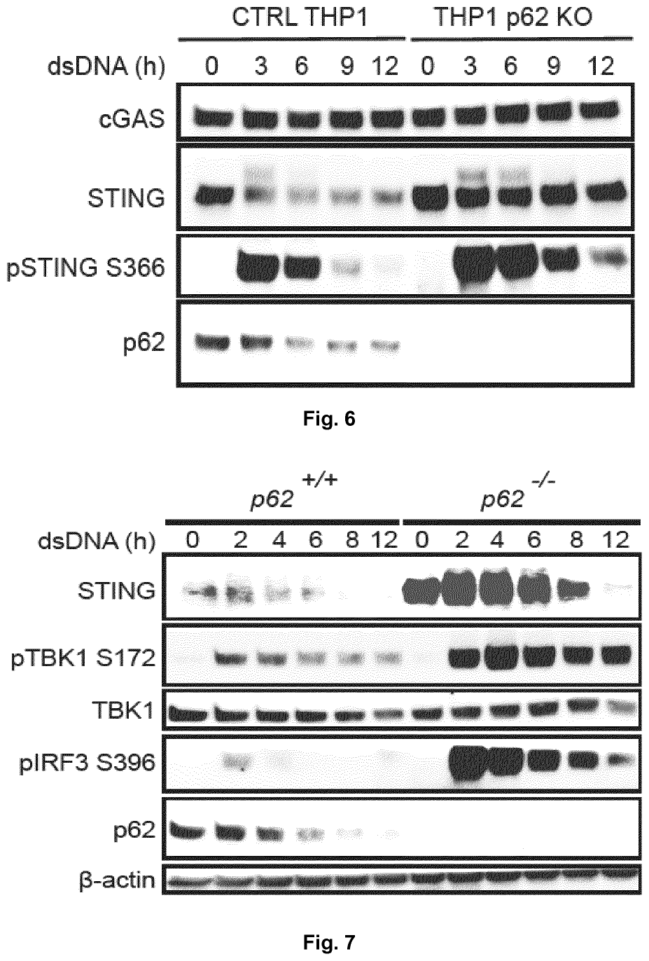

WT and P62 KO THP1 cells were stimulated with dsDNA (4 μg/ml) for 0, 3, 6, 9 and 12 hours and lysates were immunoblotted with antibodies specific for cGAS, STING, pSTING S366 and P62. It was observed that turnover of STING was abolished after DNA stimulation in P62-deficient THP1 cells (FIG. 6).

Effect of P62 on STING Degradation in MEFs Cells.

WT and p62^{-/-} MEFs cells were stimulated with dsDNA (4 μg/ml) for 0, 2, 4, 6, 8 and 12 hours and lysates were immunoblotted with antibodies specific for STING, pTBK1 S172, TBK1, pIRF3 S396, P62 and β-actin. It was observed that turnover of STING was delayed in p62^{-/-} MEFs, as well as higher basal levels of STING in p62^{-/-} MEFs (FIG. 7).

Reconstitution of wt P62 Expression in P62-Deficient MEFs and THP1 Cells.

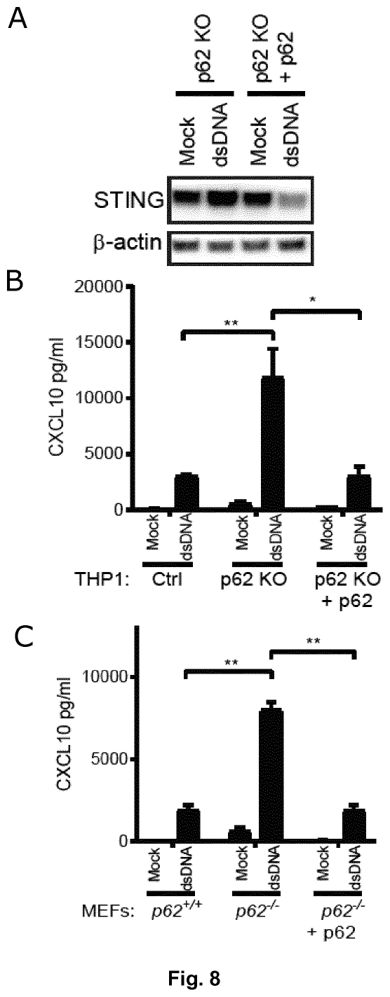

Expression of wt P62 in P62 KO THP1 cells and p62^{-/-} MEFs was reconstituted using lentiviral transduction. The cells were stimulated with dsDNA (4 μg/ml) and evaluated for levels of STING in lysates and CXCL10 in the supernatants 8 h and 16 h post-treatment, respectively. Reconstitution of wt P62 expression in P62-deficient MEFs and THP1 cells enabled these cells to degrade STING after DNA stimulation and reduced DNA-induced CXCL10 expression (FIG. 8).

Effect of P62 on Ubiquitination of STING.

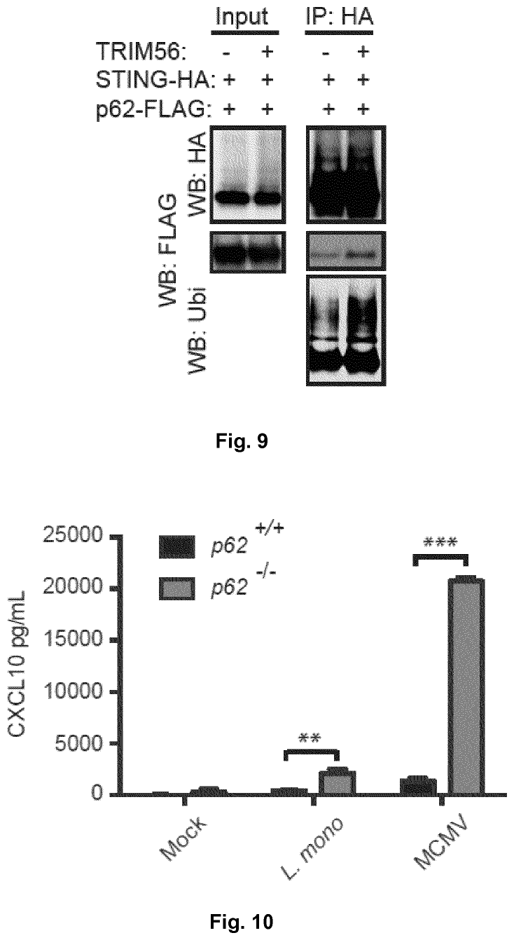

HEK293T cells were transduced with TRIM56, STING-HA, and P62-FLAG. Lysates were generated 16 h later and subjected to anti-HA immunoprecipitation. The precipitate was immunoblotted with anti-HA, anti-FLAG, and anti-ubiquitin. Elevated ubiquitination of STING was observed and the STING ubiquitination was accompanied by elevated P62-STING co-immunoprecipitation (FIG. 9).

Overall, these experiments demonstrates that P62 is essential for stimulation-induced degradation of STING, which correlates with STING ubiquitination.

### Example 4

### Regulation of Innate Immune Responses to DNA Pathogens is Dependent on P62

The experiments were performed according to the general methods as described in Example 1 above.

Effect of P62 on CXCL10 Response of MEFs Cells to DNA Pathogens.

WT and p62^{-/-} MEFs were infected with Listeria monocytogenes (MOI 25) or MCMV (MOI 10). Supernatants were harvested 18 h post treatment and lysates were isolated. Supernatants were analyzed for levels of CXCL10. Infections with the DNA pathogens stimulated elevated CXCL10 responses in the P62-deficient cells (FIG. 10).

Effect of P62 on Response of THP1 Cells to DNA Pathogens.

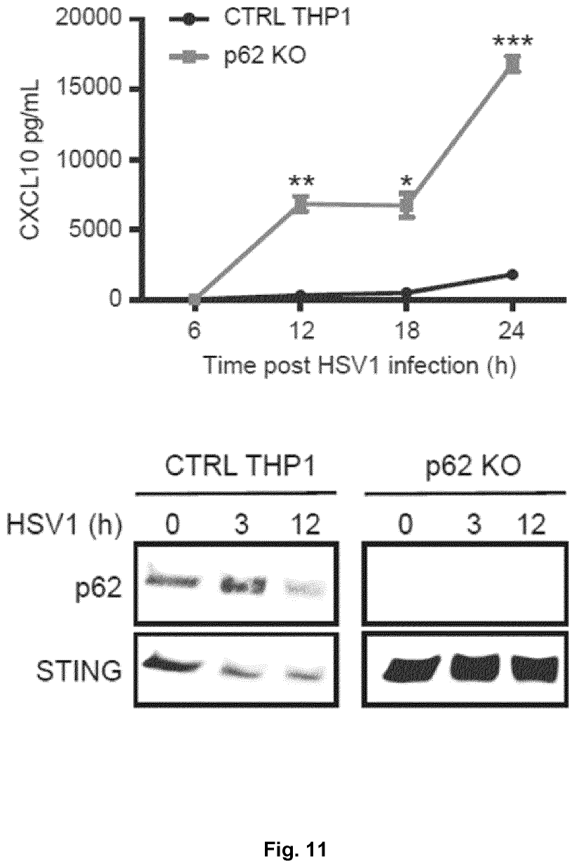

Control and P62 KO THP1 cells were infected with HSV-1 (MOI 10) for 0, 3, 6, 12, 18 and 24 hours. Supernatants were analyzed for levels of CXCL10 (A) and lysates were immunoblotted for P62 and STING (B). Infection with HSV-1 induced a strongly elevated production of CXCL10 in P62-deficient cells compared to the control cells, and STING was not degraded in the infected knockout cells (FIG. 11).

Effect of P62 on Response of Human Monocyte-Derived Macrophages to dsDNA or DNA Pathogens.

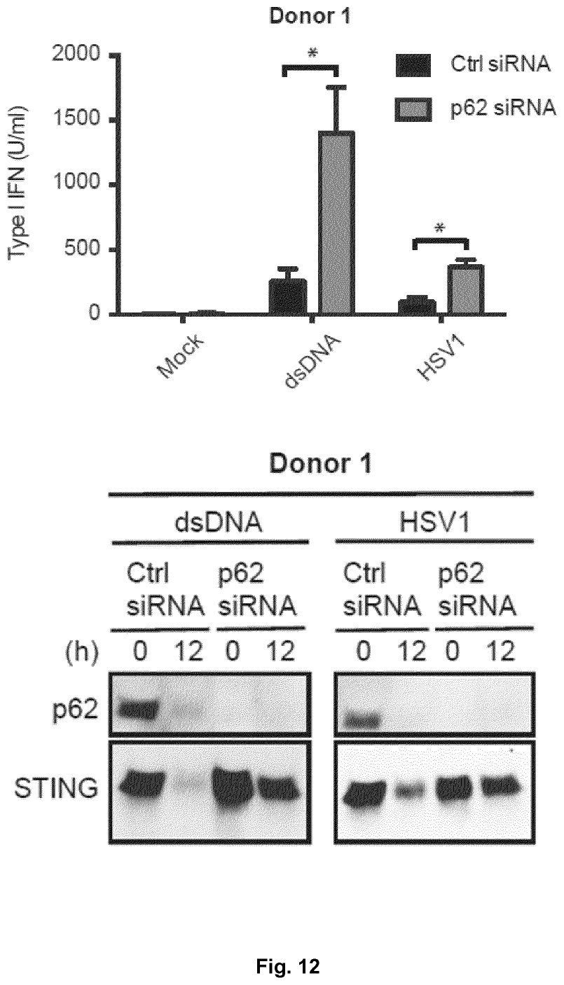

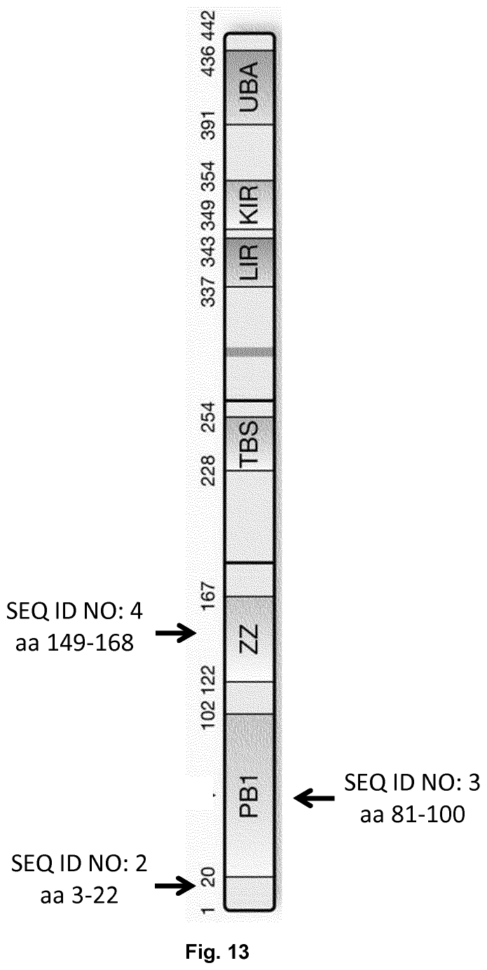

P62 was knocked down by siRNA in human monocyte-derived macrophages from 4 donors. The siRNA-treated cells were treated with dsDNA (4 μg/ml) or infected with HSV-1 (MOI 10). Supernatants were harvested 12 h post treatment and lysates were isolated at time points 0 and 12 hours. Supernatants were analyzed for levels of type I IFN, and lysates were immunoblotted for P62 and STING. A strong knockdown of P62 in the primary human macrophages was achieved, and it was observed that cells with reduced P62 expression produced more type I IFN after stimulation with DNA or infection with HSV-1. Higher constitutive levels of STING and impaired DNA-induced degradation of STING was observed (FIG. 12).

Overall, these experiments demonstrate that P62 is essential for executing degradation of STING and attenuation of signaling after infection with DNA pathogens.

### Example 5

### Peptide Modulators of P62-STING Interaction

Peptides were identified, which were able to modulate the interaction of P62 and STING and thereby modulate the levels of interferons and pro-inflammatory cytokines. The peptides are derived from P62 (FIG. 13).

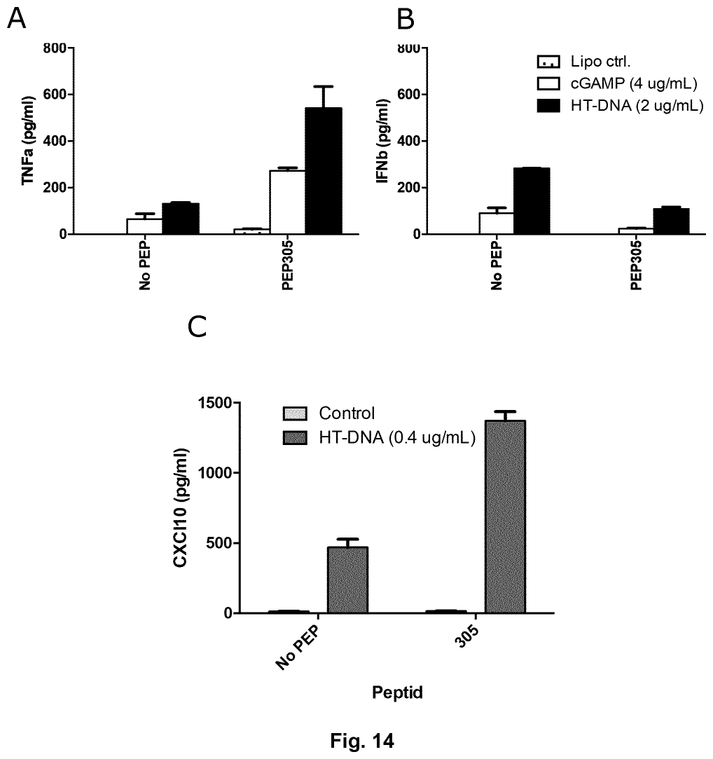

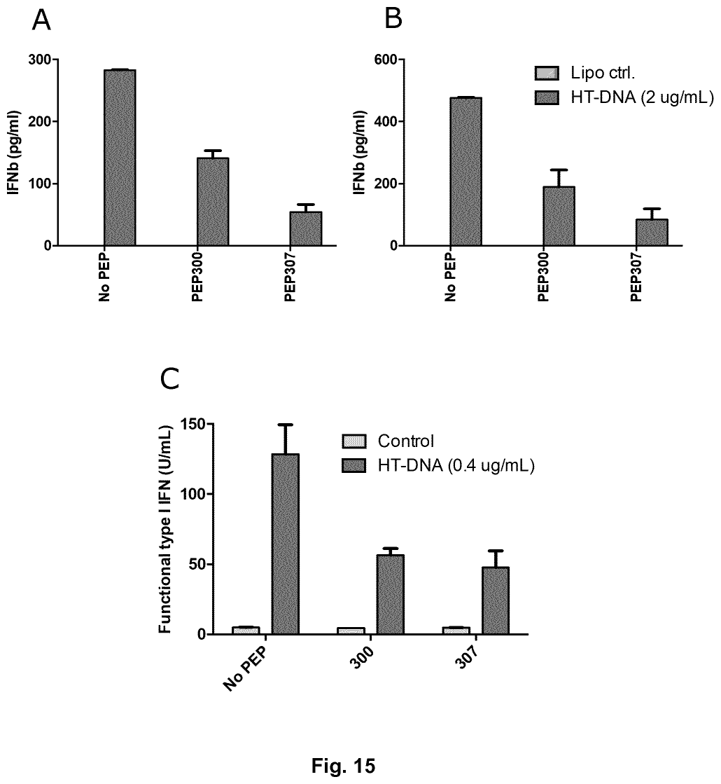

Using human PBMCs and PMA-treated THP1 cells, we identified that a peptide comprising a fragment of the PB1 domain of P62 (SEQ ID NO: 9) is very efficient in increasing inflammatory cytokine expression following stimulation with cGAMP and/or DNA, both ligands targeting the STING-pathway (FIG. 14). Peptides comprising fragments of the N-terminus (SEQ ID NO: 8) or the ZZ-domain of P62 (SEQ ID NO: 10), were both capable of reducing DNA-triggered type I interferon signaling in a STING dependent manner (FIG. 15).

Cell Culture.

Human acute monocytic leukemia cell line (THP-1) was cultured in RPMI 1640 (Lonza) supplemented with 10% heat inactivated fetal calf serum, 200 IU/mL Penicillin, 100 μg/mL Streptomycin and 600 μg/mL glutamine (hereafter termed RPMI complete). Mycoplasma infection was tested and ruled on a monthly basis using Lonza MycoAlert kit (LT07-703). To differentiate THP-1 cells into adherent phenotypically macrophages, cells were stimulated with 100 nM Phorbol 12-myristate 13-acetate (PMA, Sigma Aldrich 79346 5MG) in RPMI complete for 24 hours before medium was refreshed with normal RPMI complete and allowed to further differentiate an additional day (hereafter defined as macrophages).

Peripheral Blood Mononuclear cells (PBMCs) were isolated from healthy donors by Ficoll Paque gradient centrifugation (GE Healthcare). PBMCs were cultured in RPMI complete supplemented with IL-2.

Functional Type I IFN Assay

To quantify functional type I IFN the reporter cell line HEK-Blue™ IFN-α/β (InvivoGen) was utilized according to the manufacturer's instructions. Thirty thousand HEK-Blue cells were seeded in 96-well plates with 150 μl medium devoid of Blasticidin and Zeocin and given 50 μL supernatant the next day. This cell line expresses secreted embryonic alkaline phosphatase under the control of the IFN-α/β inducible ISG54 promotor. SEAP activity was assessed by measuring optical density (OD) at 620 nm on a micro plate reader (ELx808, BioTEK). The standard range was made with IFN-α (A2) (PBL Assay Science).

Enzyme-Linked Immunosorbent Assay

Protein levels of the cytokines CXCL10 and TNF-α in supernatants, were measured using ELISA kits from PeproTech (CXCL10; 900-T39.) and BioLegend (TNF-α; 430201.) following the manufacturer's instructions.

Stimulation

To stimulate cells with STING agonists, 2′3′cGAMP (Invitrogen) or HT-DNA was formulated with lipofectamine2000 at a final concentration of 4 μg/ml (cGAMP) and 0.4-2 μg/ml (HT-DNA).

Treatment with Peptides.

Each peptide was diluted in PBS pH 7 to a final concentration of 5 μg/μL. The peptides were then added to cell culture of PBMCs or THP1 cells at a final concentration of 10 μg/ml (THP1 cells) and 25 μg/ml (PBMCs). After 1 hour, cells were stimulated with STING agonists and supernatants collected after 20 hours and used for Type I IFN bioassay or cytokine ELISA.

Collectively, this example demonstrates that small peptides can be used for targeting the function of P62-STING interaction and can thus be used to block or boost the immunological response of the STING signaling pathway.

### Example 6

### Additional PB1 domain peptides modulating P62-STING interaction Functional Type I IFN Assay

Bioactive human type I IFN was measured on cell supernatants by use of HEK-Blue™ IFN-a/b cells as reporter cells according to the manufacturer instructions (InvivoGen).

Enzyme-Linked Immunosorbent Assay

Protein levels of the cytokines CXCL10 and TNF-α in supernatants, were measured using ELISA kits from R and D Systems (CXCL10; DY266) and (TNF-α; DY210) following the manufacturer's instructions.

Immunoblotting

Whole-cell extracts or immunoprecipitation samples were analyzed by immunoblotting. Samples were diluted in XT sample buffer and XT reducing agent and ran on a SDS—PAGE (Criterion™ TGX™). Trans-Blot Turbo™ Transfer System was used for the transfer of proteins to PVDF membranes (all reagents Bio-Rad). The membrane was blocked in 5% Difco™ skim milk (BD) or 5% bovine serum albumin (BSA) (Sigma). For detection of STING dimerization, samples were prepared for electrophoresis using non-reducing RIPA lysis buffer with 0.2% SDS and complete mini inhibitor (Sigma). The antibodies used for immunoblotting were as follows: rabbit anti-pSTING (S366) (Cell Signaling Technology, #85735), rabbit anti-pTBK1 (Ser172) (Cell Signaling, D52C2/#5483), rabbit anti-phospho p62 (Cell Signalling), Guinea pig anti-p62 (Progen), rabbit anti-STING (Cell Signalling) and mouse anti-b-actin (Abcam, AC-15/ab49900).

Surface Plasmon Resonance Analysis

For the surface plasmon resonance (SPR) analyses, BlAcore sensor chips (type CMS; Biacore, Uppsala, SE) were activated with a 1:1 mixture of 0.2 M N-ethyl-N′-(3-dimethylaminopropyl) carbodiimide and 0.05 M N-hydroxysuccinimide in water according to the manufacturer. C-terminal STING was immobilized. The SPR signal from immobilized C-terminal STING generated BlAcore resonance units (RU) that were equivalent to 66 fmol/mm2. The flow cells were regenerated with 20 μl of 1.5 M glycine-HCI (pH 3.0). The flow buffer was 10 mM HEPES, 150 mM NaCl, 1.5 mM CaCl2, and 1 mM EGTA (pH 7.4). The binding data was analyzed using the BIAevaluation program. The number of ligands bound per immobilized receptor was estimated by dividing the ratio RU ligand/mass ligand with RU receptor/mass receptor. The Kd for binding was estimated using a BIA evaluation program.

PB1-domain peptide PEP302

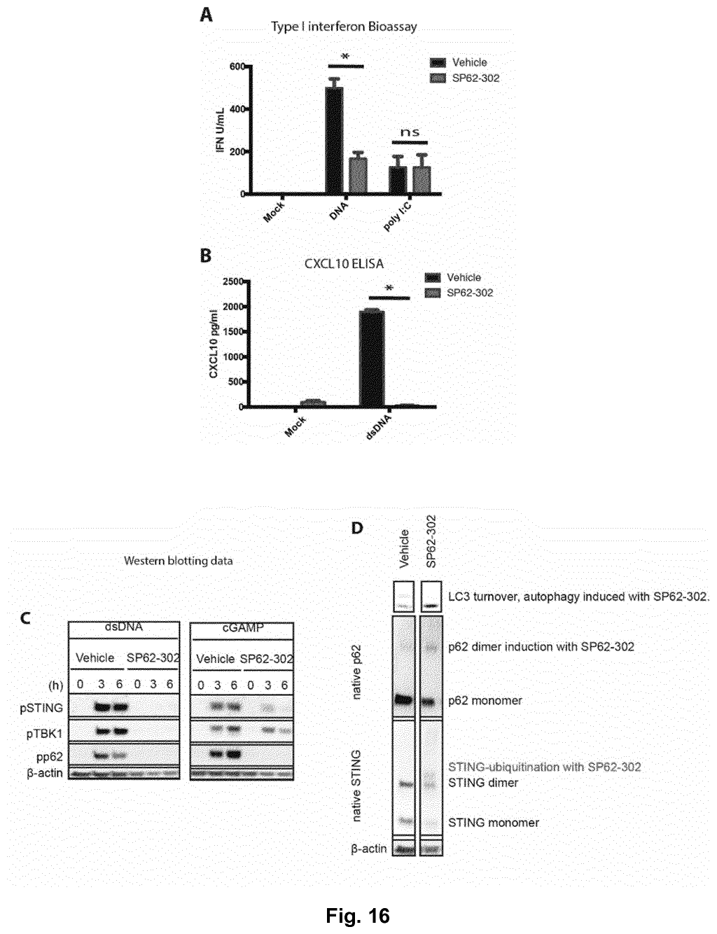

PEP302 is also derived from the p62 PB1 domain and it has immunosuppressive effects in THP1 cells.PEP302 reduces DNA-activated IFN and CXCL10 (FIG. 16A and 16B), which further indicate that the PB1 domain of p62 interacts directly with STING or regulates STING activity. Western blotting data show that PEP302 “PB1” domain can inhibit STING phosphorylation after DNA-stimulation and significantly reduce phospho-STING after cGAMP stimulation. Furthermore, phosphorylation of TBK1 and p62 is also significantly reduced in presence of PEP302 (FIG. 16C). PEP302 may induce LC3 turnover, STING- and p62-dimerization in THP1 cells treated with PEP302 (FIG. 16D). PEP302 may also induce ubiquitination of STING in THP1 cells treated with PEP302 alone.

PB1-domain peptide PEP301

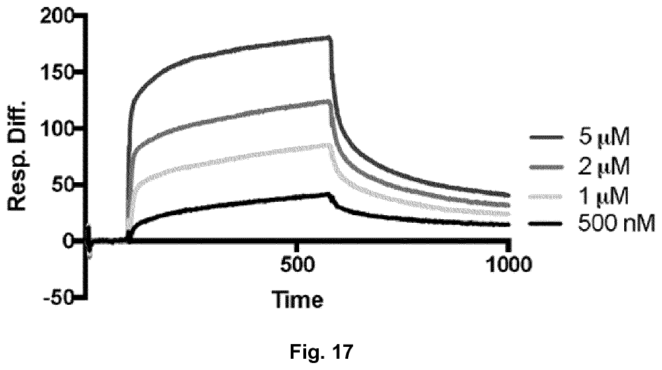

PEP301 is derived from the p62 PB1 domain, and it binds nicely to immobilized and purified C-terminal STING in Biacore analysis with Kd=746 nM (see FIG. 17).These Biacore data indicate that PB1 domain of p62 interacts with STING.

1. A compound capable of modulating the interaction between P62 and STING.

2. The compound according to claim 1, wherein the compound is capable of inhibiting or reducing the interaction between P62 and STING.

3. The compound according to claim 1, wherein the compound is capable of potentiating the interaction between P62 and STING.

4. The compound according to any one of the preceding claims, wherein said compound is capable of inducing or enhancing STING activation.

5. The compound according to any one of the preceding claims, wherein said compound is capable of inhibiting or reducing STING degradation.

6. The compound according to any one of claims 1 to 3, wherein said compound is capable of inhibiting STING activation.

7. The compound according to claim 6, wherein said compound is capable of inducing or enhancing STING degradation.

8. The compound according to any one of the preceding claims, wherein the compound is a polypeptide.

9. The compound according to any of the preceding claims, wherein said compound is a polypeptide comprising or consisting of the N-terminal domain of P62 identified as SEQ ID NO: 2 or a fragment thereof.

10. The compound according to any one of the preceding claims, wherein said compound is capable of mimicking the N-terminal domain of P62.

11. The compound according to any of the preceding claims, wherein said compound is a polypeptide comprising or consisting of the PB1 domain of P62 identified as SEQ ID NO: 3 or a fragment thereof.

12. The compound according to any one of the preceding claims, wherein said compound is capable of mimicking the PB1 domain of P62.

13. The compound according to any of claims 1 to 8, wherein said compound is a polypeptide comprising or consisting of the ZZ finger domain of P62 identified as SEQ ID NO: 4 or a fragment thereof.

14. The compound according to claim 13, wherein said compound is capable of mimicking the ZZ finger domain of P62.

15. The compound according to any of the preceding claims, wherein said compound is a polypeptide comprising or consisting of
a. the N-terminal domain of P62 (human PB1 domain) provided herein as SEQ ID NO: 2;
b. a fragment of said human PB1 domain consisting of a consecutive sequence of at least 5 amino acids of SEQ ID NO: 2; or
c. a functional homologue of the human PB1 domain sharing at least 70% sequence identity with SEQ ID NO: 2.

16. The compound according to any of the preceding claims, wherein said compound is a polypeptide comprising or consisting of
d. the PB1 domain of P62 (human PB1 domain) provided herein as SEQ ID NO: 3;
e. a fragment of said human PB1 domain consisting of a consecutive sequence of at least 5 amino acids of SEQ ID NO: 3; or
f. a functional homologue of the human PB1 domain sharing at least 70% sequence identity with SEQ ID NO: 3.

17. The compound according to any of the preceding claims, wherein said compound is a polypeptide comprising or consisting of
a. the ZZ finger domain of P62 (human ZZ finger domain) provided herein as SEQ ID NO: 4;
b. a fragment of said human ZZ finger domain consisting of a consecutive sequence of at least 5 amino acids of SEQ ID NO: 4; or
c. a functional homologue of the human ZZ finger domain sharing at least 70% sequence identity with SEQ ID NO: 4.

18. The compound according to any one of the preceding claims, wherein the compound is a polypeptide comprising or consisting of
a. a peptide according to SEQ ID NO: 5;
b. a fragment of SEQ ID NO: 5 consisting of a consecutive sequence of at least 5 amino acids of SEQ ID NO: 5; or
c. a functional homologue of SEQ ID NO: 5 sharing at least 70% sequence identity with SEQ ID NO: 5.

19. The compound according to any one of the preceding claims, wherein the compound is a polypeptide comprising or consisting of
a. a peptide according to SEQ ID NO: 6;
b. a fragment of SEQ ID NO: 6 consisting of a consecutive sequence of at least 5 amino acids of SEQ ID NO: 6; or
c. a functional homologue of SEQ ID NO: 6 sharing at least 70% sequence identity with SEQ ID NO: 6.

20. The compound according to any one of the preceding claims wherein the compound is a polypeptide comprising or consisting of
a. a peptide according to SEQ ID NO: 7;
b. a fragment of SEQ ID NO: 7 consisting of a consecutive sequence of at least 5 amino acids of SEQ ID NO: 7; or
c. a functional homologue of SEQ ID NO: 7 sharing at least 70% sequence identity with SEQ ID NO: 7.

21. The compound according to any one of the preceding claims, wherein the compound is a polypeptide comprising or consisting of
a. A peptide according to SEQ ID NO: 8;
b. a fragment of SEQ ID NO: 8 consisting of a consecutive sequence of at least 5 amino acids of SEQ ID NO: 8; or c. a functional homologue of SEQ ID NO: 8 sharing at least 70% sequence identity with SEQ ID NO: 8.

22. The compound according to any one of the preceding claims, wherein the compound is a polypeptide comprising or consisting of
a. a peptide according to SEQ ID NO: 9;
b. a fragment of SEQ ID NO: 9 consisting of a consecutive sequence of at least 5 amino acids of SEQ ID NO: 9; or
c. a functional homologue of SEQ ID NO: 9 sharing at least 70% sequence identity with SEQ ID NO: 9.

23. The compound according to any one of the preceding claims wherein the compound is a polypeptide comprising or consisting of
a. a peptide according to SEQ ID NO: 10;
b. a fragment of SEQ ID NO: 10 consisting of a consecutive sequence of at least 5 amino acids of SEQ ID NO: 10; or
c. a functional homologue of SEQ ID NO: 10 sharing at least 70% sequence identity with SEQ ID NO: 10.

24. The compound according to any one of the preceding claims wherein the compound is a polypeptide comprising or consisting of
a. a peptide according to SEQ ID NO: 11;
b. a fragment of SEQ ID NO: 11 consisting of a consecutive sequence of at least 5 amino acids of SEQ ID NO: 11; or
c. a functional homologue of SEQ ID NO: 11 sharing at least 70% sequence identity with SEQ ID NO: 11.

25. The compound according to any one of the preceding claims wherein the compound is a polypeptide comprising or consisting of
a. a peptide according to SEQ ID NO: 12;
b. a fragment of SEQ ID NO: 12 consisting of a consecutive sequence of at least 5 amino acids of SEQ ID NO: 12; or
c. a functional homologue of SEQ ID NO: 12 sharing at least 70% sequence identity with SEQ ID NO: 12.

26. The compound according to any of the preceding claims, wherein the compound is capable of binding a polypeptide comprising or consisting of
a. P62 identified as SEQ ID NO: 1, and/or
b. STING identified as SEQ ID NO: 15

27. The compound according to any one of the preceding claims, wherein said compound is a small molecule.

28. The compound according to any one of the preceding claims, wherein the compound is an antibody, an antigen-binding fragment of an antibody or a synthetic antibody.

29. The compound or polypeptide according to any one of the preceding claims, wherein said compound or polypeptide further comprise at least one conjugated moiety.

30. The compound or polypeptide according to claim 29, wherein said at least one conjugated moiety is a cell-penetrating peptide, such as Polyarginine or TAT.

31. The compound or polypeptide according to any one of the preceding claims for use as a medicament.

32. The compound or polypeptide according to any one of the preceding claims for use in the treatment of a disorder associated with STING activity.

33. The compound or polypeptide according to any one of the preceding claims for use in the treatment of a disorder associated with insufficient STING activity.

34. The compound or polypeptide according to any one of the preceding claims for use in the treatment of a disorder associated with excessive STING activity.

35. A method of treating a disorder associated with STING activity comprising administering the compound or the polypeptide according to any one of claims 1 to 30 to an individual in need thereof.

36. The compound or polypeptide for use or the method according to any one of claims 31 to 35, wherein said disorder is cancer, for example a cancer induced by chronic inflammatory signaling.

37. The compound or polypeptide for use or the method according to claim 36, wherein the cancer is a cutaneous skin tumor, for example basal cell (BCC) or squamous cell carcinoma (SCC).

38. The compound or polypeptide for use or the method according to any one of claims 31 to 35, wherein said disorder is an infection with a DNA pathogen, for example malaria or listeria.

39. The compound or polypeptide for use or the method according to any one of claims 31 to 35, wherein the disorder is an inflammatory disorder, for example psoriasis, Crohn's disease, Inflammatory bowel disease (IBD).

40. The compound or polypeptide for use or the method according to any one of claims 31 to 35, wherein the disorder is an auto-immune disease, for example Paget's disease, STING-associated vasculopathy with onset in infancy (SAVI), systemic lupus erythematosus (SLE), Aicardi-Goutieres syndrome, Sjogren's syndrome, Type 1 diabetes and multiple sclerosis.

41. The compound or polypeptide for use or the method according to any one of claims 31 to 40, wherein said treatment of said disorder further comprises administration of one or more additional active compounds.

42. The compound or polypeptide for use or the method according to claim 41, wherein the additional active compound is an anti-cancer agent.


SEQUENCE LISTING (15 sequences)

SEQ ID NO: 1 - protein, 440 aa, Homo sapiens
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 2 - protein, 19 aa, Homo sapiens
MASLTVKAYLLGKEDAARE

SEQ ID NO: 3 - protein, 83 aa, Homo sapiens
IRRFSFCCSPEPEAEAEAAAGPGPCERLLSRVAALFPALRPGGFQAHYRDEDGDLVAFSSDEELTMAMSYVKDDIFRIYIKEK

SEQ ID NO: 4 - protein, 46 aa, Homo sapiens
VHPNVICDGCNGPVVGTRYKCSVCPDYDLCSVCEGKGLHRGHTKLA

SEQ ID NO: 5 - protein, 20 aa, Homo sapiens
SLTVKAYLLGKEDAAREIRR

SEQ ID NO: 6 - protein, 20 aa, Homo sapiens
EELTMAMSYVKDDIFRIYIK

SEQ ID NO: 7 - protein, 20 aa, Homo sapiens
DLCSVCEGKGLHRGHTKLAF

SEQ ID NO: 8 - protein, 32 aa, Homo sapiens
SLTVKAYLLGKEDAAREIRRGRKKRRQRRRPQ

SEQ ID NO: 9 - protein, 32 aa, Homo sapiens
EELTMAMSYVKDDIFRIYIKGRKKRRQRRRPQ

SEQ ID NO: 10 - protein, 32 aa, Homo sapiens
DLCSVCEGKGLHRGHTKLAFGRKKRRQRRRPQ

SEQ ID NO: 11 - protein, 32 aa, Homo sapiens
FSFCCSPEPEAEAEAAAGPGGRKKRRQRRRPQ

SEQ ID NO: 12 - protein, 32 aa, Homo sapiens
PGPCERLLSRVAALFPALRPGRKKRRQRRRPQ

SEQ ID NO: 13 - DNA, 20 bp, Artificial sequence
  Guide RNA (gRNA)
  misc_feature (1)..(20): P62, guideRNA #1
gatggccatgtcctacgtga

SEQ ID NO: 14 - DNA, 20 bp, Artificial sequence
  Guide RNA (gRNA)
  misc_feature (1)..(20): P62 guideRNA #2
gtcatccttcacgtaggaca

SEQ ID NO: 15 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS